(19) United States
(12) Patent Application Publication
(10) Pub. No.: US 2014/0030199 A1
(43) Pub. Date: Jan. 30, 2014

(54) ARTIFICIAL NAIL COMPOSITION

(71) Applicant: SHOFU INC., Kyoto-shi (JP)

(72) Inventors: Akiko Matsumoto, Kyoto-shi (JP); Hisaki Tanaka, Kyoto-shi (JP); Yutaka Yamaguchi, Kyoto-shi (JP); Toshiyuki Nakatsuka, Kyoto-shi (JP)

(73) Assignee: SHOFU INC., Kyoto-shi (JP)

(21) Appl. No.: 13/770,514

(22) Filed: Feb. 19, 2013

(30) Foreign Application Priority Data
Jul. 24, 2012 (JP) 2012-164216

(51) Int. Cl. A61K 8/37; A61Q 3/02

(52) U.S. Cl. CPC A61K 8/37; A61Q 3/02
USPC 424/61

(57) ABSTRACT

An artificial nail composition having improved adhesion with natural nails (user's own nails) is provided.

The present invention relates to an artificial nail composition comprising (a) a photo-polymerizable monomer having at least one primary alcoholic hydroxyl group in the molecule, and/or a photo-polymerizable monomer having at least one carboxyl group and at least one primary alcoholic hydroxyl group in the molecule; (b) a photo-polymerizable monomer having at least one carboxyl group in the molecule except for the photo-polymerizable monomer (a); (c) a photo-polymerizable monomer except for the photo-polymerizable monomers (a) and (b); and (d) a photo-polymerization initiator.

### BACKGROUND OF THE INVENTION

1. Field of the Invention

The present invention relates to a novel artificial nail composition.

2. Description of the Related Art

Artificial nail compositions are the materials for farming artificial nails generally called as gel nails and are used to impart special aesthetic appearance to natural nails (user's own nails) by applying, polymerizing and curing the compositions on natural nails as well as to extend natural nails. In addition, the color gel nails containing a pigment may be used for nail art, formation of artificial nails and the like.

A basic method for applying gel nails has been generally known as natural nail gel floater in which gel nails are applied on natural nails, and then polymerized and cured to impart special aesthetic appearance. Alternatively, gel nail materials may be sometimes applied on nail tips.

The general application method, natural nail gel floater, usually follows the following steps in series:
(Step 1) Sand the surface of nails with a sponge nail file;
(Step 2) Clean off oil and dusts with a wiper soaked with a nail cleanser;
(Step 3) Apply clear gel (artificial nail composition) on the whole natural nail;
(Step 4) Carry out photo-polymerization using a photo-polymerization apparatus;
(Step 5) On the polymerized artificial nail composition, apply color or nail art using color gel (artificial nail composition);
(Step 6) Carry out photo-polymerization using a photo-polymerization apparatus;
(Step 7) Repeat steps 5 and 6, if necessary;
(Step 8) Apply top gel (artificial nail composition) on the whole natural nail;
(Step 9) Carry out photo-polymerization using a photo-polymerization apparatus; and
(Step 10) Clean off uncured gel (artificial nail composition) on the surface with a wiper soaked with a gel remover or cleanser.

Gel nail materials have improved adhesion durability, aesthetic appearance and the like compared to a general nail cosmetic, manicure (also referred to as nail polish or nail lacquer) and are applied relatively easily as described above. Therefore, they are used for one of main treatments in nail salons and the like.

However, it has been a problem that artificial nail compositions applied at fingertips may be detached or lost due to various loads of everyday life. It is believed that such detachment or loss of artificial nail composition, due to low adhesion between natural nails and artificial nails causes not only aesthetic discomfort but also infectious diseases in artificial nail users mainly exemplified as green nail. Thus, there is a need for development in artificial nail compositions having superior adhesion.

The present inventors proposed an artificial nail composition comprising (a) a compound having at least one radical polymerizable unsaturated double bond in the molecule, (b) an acidic phosphorus compound having at least one radical polymerizable unsaturated double bond in the molecule, and (c) a radical polymerization initiator (Japanese Patent Application Publication No. 2010-53097). However, there is still room for further improvement in a product life or adhesion of the artificial nail composition containing (b) an acidic phosphorus compound having at least one radical polymerizable unsaturated double bond in the molecule.

Japanese Patent Application Publication No. 2002-505S15 suggests an artificial nail composition comprising a monoethylenically unsaturated vinyl monomer that contains two or more carbonyl groups and one or more addition-polymerizable, ethylenically unsaturated monomers for improved adhesion to natural nails. However, adhesion for this artificial nail composition is still not sufficient.

Japanese Patent Application Publication No. 2009-126833 discloses an artificial nail composition cured with ultraviolet irradiation characterized in that it contains an ionic monomer polymerizable with ultraviolet irradiation. However, this invention is to improve biological safety of removal of the artificial nail composition after application and therefore adhesion of the composition to natural nails is not considered in the invention.

Japanese Patent Publication No. 3208519 suggests a protective agent and protective agent kit tor nails characterized in that they contain a primer containing a methacryloyl group in a molecule as a main component. Japanese Patent Publication No. 3208519 discloses that, as a result of investigation and test on various agents adhesive to nail protein (collagen), a primer of a high molecular compound which has been used intraorally for dental purposes (dental implants, coatings etc) and has been proved to be safe for human use can be used as a satisfactory protective agent for nails by applying and curing the primer on the surface of nails before polishing the surface of the cured primer. However, this invention consistently relates to the protective agent for nails, which is a material intended to prevent deformation of nails of hands and feet due to illness and external forces, and to restore nails to their original forms. Namely, Japanese Patent Publication No. 3208519 does not provide any investigation for makeup materials providing special aesthetic appearance to natural nails.

Japanese Patent Application Publication No. H2-19313 discloses a manicure composition effective for support and protection of nails including natural and artificial nails and curable under ultraviolet after being applied to finger nails of hands and feet, which comprises an aliphatic urethane acrylate and tripropylene glycol, diacrylate, trimethylolpropane ethoxylate triacrylate, methacrylic acid, 1-hydroxycyclohexyl phenyl ketone and butyl acetate. In this invention, a primer containing an alkylsilane alkoxide is used for adhesion between an artificial nail (plastic nail) and the manicure composition. However, adhesion is still not sufficient for this composition.

Japanese Patent Application Publication No. 2010-75366 discloses that an artificial nail composition comprising a compound having a radical polymerizable unsaturated double bond and anti-bacterial group or an anti-bacterial filler has anti-bacterial properties. However, again, the artificial nail composition in Japanese Patent Application Publication No. 2010-75366 is not investigated for its adhesion to user's own nails.

### SUMMARY OF THE INVENTION

Accordingly, a main objective of the present invention is to provide an artificial nail composition having improved adhesion to natural nails (user's own nails).

The present inventors have carried out extensive studies in view of the problems accompanying with the conventional art and as a result found that a specific composition can achieve the above objective, thereby completing the present invention.

Thus the present invention is directed to the following artificial nail composition.
1. An artificial nail composition comprising (a) a photo-polymerizable monomer having at least one primary alcoholic hydroxyl group in the molecule, the photo-polymerizable monomer including a photo-polymerizable monomer having at least one carboxyl group in the molecule; (b) a photo-polymerizable monomer having at least one carboxyl group in the molecule except for the photo-polymerizable monomer (a); (c) a photo-polymerizable monomer except for the photo-polymerizable monomers (a) and (b); and (d) a photo-polymerization initiator.
2. The artificial nail composition according to the above item 1, comprising 5 to 50 parts by weight of the photo-polymerizable monomer (a), 1 to 25 parts by weight of the photo-polymerizable monomer (b), 20 to 93.5 parts by weight of the photo-polymerizable monomer (c) and 0.5 to 5 parts by weight of the photo-polymerization initiator (d), the total of the components (a), (b), (c) and (d) being 100 parts by weight.
3. The artificial nail composition according to the above item 1 or 2, which is liquid.
4. The artificial nail composition according to any of the above items 1 to 3, which is free from an organic solvent.
5. The artificial nail composition according to any of the above items 1 to 4, which is used for formation of an artificial nail on a surface of a natural nail.
6. The artificial nail composition according to the above item 5, wherein a method for the formation comprises the steps of (1) applying the liquid artificial nail composition on the surface of the natural nail to form a coating film and (2) irradiating light to the coating film to polymerize the photo-polymerizable monomers.

### ADVANTAGES OF THE INVENTION

According to the present invention, an artificial nail composition having improved adhesive property to natural nails (user's own nails) can be provided. Accordingly, detachment or loss of artificial nails can be effectively prevented and as a result aesthetic appearance of artificial nails can foe retained for relatively prolonged period of time.

The present artificial nail composition having the above features is useful not only for morphology formation of artificial nails (extension, shaping of nails) but also for drawing a design called nail art, for imparting strength to fragile natural nails or regenerating or protecting deformed or discolored nails for the purpose of medical support, for example.

### DESCRIPTION OF THE PREFERRED EMBODIMENTS

### 1. Artificial Nail Composition

The artificial nail composition of the present invention (the present composition) is characterized in that it contains (a) a photo-polymerizable monomer having at least one primary alcoholic hydroxyl group in the molecule, and/or a photo-polymerizable monomer having at least one carboxyl group and at least one primary alcoholic hydroxyl group in the molecule (hereinafter also referred to as the component (a)); (b) a photo-polymerizable monomer having at least one carboxyl group in the molecule except for the photo-polymerizabie monomer (a) (hereinafter also referred to as the component (b)); (c) a photo-polymerizable monomer except for the photo-polymerizable monomers (a) and (b) (hereinafter also referred to as the component (c)); and (d) a photo-polymerization initiator (hereinafter also referred to as the component (d)).

One of the features of the present composition is, particularly, that it contains three different photo-polymerizable monomers, i.e. the components (a) to (c). These photo-polymerizable monomers have polymerizable functional groups. The polymerizable functional group is not limited and may include, for example, a methacryloyl group, an acryloyl group, an allyl group, a vinyl group and the like. Among these, the compound having at least one polymerizable functional group of an acryloyl group and a methacryloyl group can be suitably used for the present invention. “(Meth)acryloyl” or “(meth)acrylate” as used herein collectively represents both acryloyl group-containing photo-polymerizable monomers and methacryloyl group-containing photo-polymerizable monomers.

The photo-polymerizable monomers of the components (a) to (c) may be the monomers described below as well as their oligomers, polymers and the like as long as they have the polymerizable functional group (s). The oligomers or polymers preferably have the weight average molecular weight of around 400 to 55,000. When the weight average molecular weight is within this range, the present composition can be provided with better adhesion, curing properties and the like without impairing its reactivity.

Component (a)

The component (a) used is the photo-polymerizable monomer having at least one primary alcoholic hydroxyl group in the molecule. The component (a) also includes a photo-polymerizable monomer having at least one carboxyl group as well as at least one primary alcoholic hydroxyl group in the molecule. Namely, according to the present composition, a photo-polymerizable monomer having at least one primary alcoholic hydroxyl group as well as at least one carboxyl group in the molecule belongs to the component (a), not to the component (b) described below.

The photo-polymerizable monomer which can be used as the component (a) is not specifically limited and may include, for example, monohydroxyalkyl (meth)acrylates such as 2-hydroxyethyl (meth)acrylate, 3-hydroxypropyl (meth)acrylate, 4-hydroxybutyl (meth)acrylate, 5-hydroxypentyl (meth)acrylate, 6-hydroxyhexyl (meth) acrylate, 10-hydroxydecyl (meth)acrylate; dihydroxyalkyl (meth)acrylates such as 1,2- or 1,3-dihydroxypropyl (meth)acrylate; (meth)acrylates of polyalkylene glycols such as diethylene glycol mono(meth)acrylate, triethylene glycol mono(meth)acrylate, pentaethylene glycol mono(meth)acrylate, polyethylene glycol mono(meth)acrylate, dipropylene glycol mono (meth) acrylate; hydroxyl group-containing (meth) acrylamides such as N-(1,3-dihydroxypropyl) (meth)acrylamide and the like. One or two or more of these components (a) may be used. Among these, at least one of 2-hydroxyethyl (meth)acrylate and 2-hydroxypropyl (meth)acrylate is suitable.

In the present composition, the component (a) may also have, in addition to the primary alcoholic hydroxyl group(s), at least one functional group such as a carboxyl group, a phosphate group, a sulfonic acid group, an amino group, a glycidyl group in the molecule.

The amount of the component (a) may be appropriately selected depending on the type of the component (a) used; however, it is usually preferably 5 to 50 parts by weight and particularly more preferably 5 to 20 parts by weight with the total of the components (a) to (d) being 100 parts by weight. When the content is within this range, the composition can be effectively provided with superior adhesion as well as appropriate viscosity and mechanical strength desirable for artificial nail compositions.

### Component (b)

The component (b) used is the photo-polymerizable monomer having at least one carboxyl group in the molecule. The component (b) excludes the photo-polymerizable monomer (a), Accordingly, when the component (a) employed is a photo-polymerizable monomer having a primary alcoholic hydroxyl group and a carboxyl group in the molecule, the component (b) is used in addition to this monomer. In other words, the component (b) may be a photo-polymerizable monomer having at least one carboxyl group without any primary alcoholic hydroxyl group in the molecule.

The photo-polymerizable monomer which can be used as the component (b) is not specifically limited and may include, for example, (meth)acrylate ester compounds of α-β unsaturated carboxylic acids such as acrylic acid, methacrylic acid, maleic acid; vinyl aromatic compounds such as 4-vinyl benzoic acid; carboxylic compounds having a linear hydrocarbon group between a (meth) acryloyloxy group and a carboxylic group such as
11-(meth)acryloyloxy-1, 1-undecane dicarboxylic acid;
   (meth)acryloyloxyalkyl naphthalene (poly)carboxylic acids such as
6- (meth)acryloyloxyethyl naphthalene-1,2,6-tricarboxylic acid;
   (meth)acryloyloxyalkyl trimellitic acids such as
4-(meth)acryloyloxyalkyl trimellitic acids,
4-(meth)acryleyloxymethyl trimellitic acid,
4-(meth)acryloyloxyethyl trimellitic acid,
4-(meth)acryloyloxybutyl trimellitic acid; hydroxyl group-containing compounds such as
4-[2-hydroxy-3-(meth)acryloyloxy]butyl trimellitic acid;
   carboxybenzoyloxy-containing compounds such as
2,3-bis (3,4-dicarboxybenzoyloxy)propyl (meth) acrylate; N- and/or
O-substituted mono- or di- (meth)acryloyloxy amino acids such as
N,O-di(meth)acryloyloxytyrosine, O-(meth)acryloyloxytyrosine,
N-(meth)acryloyloxytyrosine, N-(meth)acryloyloxyphenylalanine,
O-(meth)acryloyloxyphenylalanine,
N,O-di(meth)acryloyloxyphenylalanine; N- and/or O-mono- or
di-(meth) acryloyl (amino or oxy) benzoic acid-based compounds such as N-(meth)acryloyl-4-aminobenzoic acid,
N-(meth)acryloyl-5-aminobenzoic acid, 2-, 3- or
4-(meth)acryloyloxybenzoic acid, 4- or
5-(meth)acryloylaminosalicylic acid; addition reaction products of a hydroxyalkyl (meth)acrylate and an unsaturated polycarboxylic anhydride such as an addition product of 2-hydroxyethyl (meth)acrylate and pyromellitic dianhydride, an addition product of 2-hydroxyethyl (meth)acrylate and maleic anhydride,
3,3′, 4,4 ′-benzophenone tetracarboxylic dianhydride or
3,3′,4′-biphenyl tetracarboxylic dianhydride;
polycarboxybenzoyloxy- and (meth)acryloyloxy-containing compounds such as 2-(3, 4-dicarboxybenzoyloxy)-1,3-di(meth)acryloyloxypropane; an addition product of
N-phenylglycine or N-tolylglycine and glycicdyl (meth)acrylate;
N-alkyl-N-(hydroxy(meth)acryloyloxyalkyl) amino phthalic acid compounds such as
4-[N-(2-hydroxy-3-(meth)acryloyloxypropyl)amino]phthalic acid,
3- or 4-[N-methyl-N-(2-hydroxy-3-(meth)acryloyloxypropyl)amino]phthalic acid and the like. One or two or more of these components (b) may be used. Among these, at least one of methacrylic acid, (meth)acrylate ester compounds of α-unsaturated carboxylic acids, (meth) acryloyloxyalkyl trimellitic acids and anhydrides thereof and carboxypolycaprolactone (n=1.8) mono(meth)acrylate is suitable.

In the present composition, the component (b) may also have, in addition to the carboxyl group, at least one functional group such as a phosphate group, a sulfonate group, an amino group, a glycidyl group in the molecule.

The amount of the component (b) may be appropriately selected depending on the type of the component (b) used; however, it is usually preferably 1 to 25 parts by weight and particularly more preferably 2 to 20 parts by weight with the total of the components (a) to (d) being 100 parts by weight. When the content is within this range, the composition can be effectively provided with superior adhesion as well as appropriate viscosity and mechanical strength desirable for artificial nail compositions.

### Component (c)

The component (c) used is a photo-polymerizable monomer which does not belong to any of the components (a) and (b). Namely, the component (c) used is a photo-polymerizable monomer which does not have alcoholic hydroxyl group or carboxyl group in the molecule.

The photo-polymerizable monomer which can be used as the component (c) is not specifically limited and examples thereof may include the following compounds classified according to the number of the polymerizable functional group, for example.

The compound having one polymerizable functional group may include, for example, methoxy ethylene glycol (meth)acrylate, methoxy polyethylene glycol (meth)acrylate, phenoxy ethylene glycol (meth)acrylate, phenoxy polyethylene glycol (meth)acrylate, 2-hydroxy-3-phenoxypropyl (meth)acrylate, 2-(meth)acryloyloxy ethyl succinate, 2-(meth)acryloyloxy ethyl phthalic acid, 2-(meth)acryloyloxy propyl hexahydrophthalic acid, stearyl (meth)acrylate, 3-chloro-2-hydroxypropyl (meth)acrylate, isobornyl (meth)acrylate and the like.

The compound having two polymerizable functional groups may include, for example, 1, 6-hexanediol di(meth)acrylate, 1,9-nonanediol di(meth)acrylate, 1,10-decanediol di(meth)acrylate, neopentyl glycol di(meth)acrylate, 2-methyl-1,8-octanediol di(meth)acrylate, glycerin di(meth)acrylate, ethylene glycol di(meth)acrylate, polyethylene glycol di(meth)acrylate, propylene glycol di(meth)acrylate, polypropylene glycol di(meth)acrylate, ethoxylated polypropylene glycol di(meth)acrylate, ethoxylated propylene glycol di(meth)acrylate, ethoxylated bisphenol A di(meth)acrylate, propoxylated bisphenol A di(math)acrylate, propoxylated ethoxylated bisphenol A di(meth)acrylate, tricyclodecane dimethanol di(meth)acrylate and the like.

The compound having three or more polymerizable functional groups may include, for example, trimethylolpropane tri(meth)acrylate, ethoxylated trimethylolpropane tri(meth)acrylate, ethoxylated glycerin tri(meth)acrylate, ditrimethylol propane tetra(meth)acrylate, pentaerythritol tri(meth)acrylate, pentaerythritol tetra(meth)acrylate, propoxylated pentaerythritol tetra(meth)acrylate, ethoxylated pentaerythritol (meth)acrylate, dipentaerythritol hexa(meth)acrylate, ethoxylated isocyanuric acid tri (meth)acrylate and the like.

One or two or more of these components (c) can be used. For example, when a certain component (c) has very high viscosity or is solid at room temperature, another component (c) which is a photo-polymerizable monomer having low viscosity can be preferably mixed to the certain component (c). For example, the present composition can be used as a liquid composition without using an organic solvent by appropriately selecting the photo-polymerizable monomer(s).

The component, (c) used may be a photo-polymerizable monomer other than the above photo-polymerizable monomers, e.g. an oligomer or polymer having at least one polymerizable functional group in the molecule according to the purpose of the artificial nail composition. The component (c) may also have a substituent such as an acidic group, a fluoro group and the like in the same molecule. The oligomer having at least one polymerizable functional group in the molecule may include, for example, known oligomers such as urethane oligomers, epoxy oligomers, acrylic oligomers, polyester oligomers and particularly urethane (meth)acrylate oligomers can be preferably used. The oligomers or polymers preferably have the weight average molecular weight of 400 to 55,000, as similar to the case described above. When the weight average molecular weight is within this range, the present composition can be provided with better adhesion, curing properties and the like without impairing its reactivity.

One or two or more of these components (c) may be used. For example, when a certain component (c) has very high viscosity or is solid at room temperature, another component (c) which is a photo-polymerizable monomer having low viscosity may be suitably mixed in combination.

The amount of the component (c) may be appropriate selected depending on the type of the component (c) used; however, it is usually preferably 20 to 93.5 parts by weight and particularly more preferably 50 to 90 parts by weight with the total of the components (a) to (d) being 100 parts by weight. When the content is within this range, the composition can be effectively provided with appropriate viscosity and mechanical strength desirable for artificial nail compositions as well as with thermal properties.

### Component (d)

The component (d) may be a known or commercially available photo-polymerization initiator and include, for example, benzoin ethers, benzyl ketals, α-dialkoxy acetophenones, α-hydroxy alkylphenones, α-aminoalkylphenones, acylphosphine oxides, benzophenones, thioxanthones, titanocenes and the like. Among these, at least one of α-hydroxy alkylphenones and acylphosphine oxides can be suitably used for the present invention.

The amount of the component (d) may be appropriately selected depending on the type of the component (d) used; however, it is usually preferably 0.5 to 5 parts by weight and particularly more preferably 0.5 to 3 parts by weight with the total of the components (a) to (d) being 100 parts by weight. When the content is within this range, the composition can be effectively provided with superior photo-curing properties.

In the present invention, the photo-polymerization initiator may also be preferably used with a photo-polymerization accelerator in combination. The photo-polymerization accelerator is particularly preferably tertiary amines which may specifically include N,N-dimethylaniline, N,N-diethylaniline, N, N-di-n-butylaniline, ISf,N-dibenzylaniline, N,N-dimethyl-p-toluidine, N,N-dimethyl-m-toluidine, N,N-diethyl-p-toluidine, p-bromo-N,N-dimethylaniline, m-chloro-N,N-dimethylaniline, p-dimethylaminobenzaldehyde, p-dimethylaminoacetophenone, p-dimethylaminobenzoic acid, p-dimethylaminobenzoic acid ethyl ester, p-dimethylaminobenzoic acid amino ester, N,N-dimethylanthranilic acid methyl ester, N,N-dihydroxyethylaniline, N,N-dihydroxyethyl-p-toluidine, p-dimethylaminophenyl alcohol, p-dimethylaminostyrene, N,N-dimethyl-3,5-xylidine, 4-dimethylaminopyridine, N, N-dimethyl-α-naphtylamine, N,N-dimethyl-β-naphthylamine, tributylamine, tripropylamine, triethylamine, N-methyldiethanolamine, N-ethyldiethanolamine, N,N-dimethylhexylamine, N,N-dimethyldodecylamine, N,N-dimethylstearylamine, N, N-dimethylaminoethyl methacrylate, N,N-diethylaminoethyl metharylate, 2, 2′-(n-butylimino) diethanol and the like.

The amount of the photo-polymerization accelerator to be added, if any, may be appropriately selected depending on the type of the photo-polymerization accelerator used; however, it is usually preferably 0.1 to 5.0 parts by weight and particularly more preferably 0.1 to 2.0 parts by weight with the total of the components (a) to (d) being 100 parts by weight. When the content is within this range, the composition can foe provided with superior surface or inner curing properties as well as low yellowing properties.

### Other Components

The present composition may include, other than the components (a) to (d), additives which are optionally added to known artificial nail compositions at a range without deteriorating effects of the present invention. The additives may include, for example, colorants (pigments etc.), polymerization inhibitors, anti-discoloration agents, fluorescence agents, ultraviolet absorbing agents, antibacterial agents, preservatives and the like. Particularly, the present composition is preferably free from an organic solvent in view of working environments and the like.

The proportion of the total amount of the components (a) to (d) in the present composition is not limited; however, it may be usually 80 to 100% by weight, preferably 90 to 100% by weight and more preferably 95 to 100% by weight in the present composition.

### Nature of the Present Composition

The nature of the present composition is not limited; however it is usually provided as a liquid composition. Thus, the composition can be used which contains a component in liquid form at ambient temperatures (particularly at 25° C.) and under ordinary pressure. This nature can be obtained by using at least one of the components (a) to (d) which is in liquid form at ambient temperatures and under ordinary pressure.

The viscosity (at 25° C.) of the present composition when it is in liquid form is not particularly limited; however, generally, it may be appropriately selected within the range of around 1.0 to 100 [Pa·s]. When the viscosity is within this range, the composition can achieve further superior coating properties, workability and the like.

### 2. Preparation of the Present Composition

The present composition can be prepared by homogeneously mixing the above components. The method for mixing is not limited and may be carried out with known mixing/stirring devices such as a mixer or a kneader.

It is necessary to store the present composition in a dark room during and after the preparation when it polymerizes and cures under irradiation of light (LED, UV etc.) at the point of use.

### 3. Use of Artificial Nail Composition

The present composition can be suitably used for formation of artificial nails on the surface of natural nails (user's own nails). More specifically, the present composition can be used in a method for forming an artificial nail comprising the steps of
(1) applying the liquid artificial nail composition on the surface of the natural nail to form a coating film (application step) and
(2) irradiating light to the coating film to polymerize the photo-polymerizable monomers (curing step).

In the above application step, the coating film is formed by applying the liquid artificial nail composition on the surface of a natural nail. Application may be carried out under the conditions according to known methods. According to the present invention, known pre-treatment such as sanding the surface of nails, cleaning off oil and dusts and the like may be carried out before the application step.

In the above curing step, the photo-polymerizable monomers are polymerized by irradiating light to the coating film. Namely, it is preferable to polymerize the photo-polymerizable monomers in the present composition under irradiation of light (preferably LED). The wavelength of light for irradiation is not limited; however, it is preferably within the range of 360 to 500 nm, usually. Any known or commercially available light source can be used for irradiating light with such a wavelength, which for example includes LED irradiating apparatuses, UV irradiating apparatuses and the like. The light source used for the present invention is desirably a LED irradiating apparatus. The LED irradiating apparatus is desirable because it can be downsized and light having high power can be easily irradiated.

### Examples

The features of the present invention are now described in more detail by way of the following Examples and Comparative examples. However, it should be recognized that the scope of the invention is not limited to these examples.

### Components and Their Abbreviations Used for Preparation of Artificial Nail Compositions in Examples

### (c) Photo-Polymerizable Monomer

PUDA: Urethane diacrylate oligomer (Molecular weight: 15,000)

IEMA: Isobornyl methacrylate

TEGDMA: Triethylene glycol dimethacrylate

(a) Photo-Polymerizable Monomer having at Least One Primary Alcoholic Hydroxyl Group in the Molecule

2HEMA: 2-Hydroxyethyl methacrylate

2HPMA: 2-Hydroxypropyl methacrylate

(a′) Photo-Polymerizable Monomer having at Least One Secondary Alcoholic Hydroxyl Group in the Molecule

GM: Glycerol dimethacrylate

(b) Photo-Polymerizable Monomer having at Least One Carboxyl Group in the Molecule

MA: Methacrylic acid

4META.: 4-Metbacryloyloxyethyl trimellitic acid anhydride

A-SA: 2-Acryloyloxyethyl succinate

A-PCLA: Carboxypolycaprolactone (n=1.8) monoacrylate

### (d) Photo-Polymerization Initiator

MAPO: Diphenyl (2, 4,6-trimethylbenzoyl)phosphine oxide

BP: Benzophenone

2HMPP: 2-Hydroxy-2-methylpropiophenone

### Examples 1 to 15 and Comparative Examples 1 to 6

According to the proportions shown in Tables 1 to 3, respective components were mixed in a revolution/rotation mixer at 23° C. under atmospheric pressure to prepare artificial nail compositions in homogeneous liquid form. The prepared artificial nail compositions (about 10 g) were filled in black light-protecting jars to be used for the following respective Test examples.

### Test Example 1

Adhesion test was carried out for the artificial nail compositions obtained in Examples and Comparative examples. The test method was as follows.

The shear bonding test was carried out in which a Nylon-6 flat plate (15.0-mm length×15.0-mm width×3.0-mm thickness) was used as a substitute for a natural nail which is an adherend in this test. The Nylon-6 fiat plate was polished with waterproof abrasive paper (No. 1200) under running water and subjected to ultrasonic cleaning and drying to prepare an adherend. On the adherend surface was applied a piece of a double-faced adhesive tape having a hole with a diameter of 5 mm in order to define the adhesion area. A plastic mold having 5-mm diameter×2-mm height was then fixed on the defined adhesion surface and the artificial nail composition was filled in the mold. Light was irradiated for 240 seconds with a commercially available lamp for gel nails (Presto LED Lamp) in order to polymerize the artificial nail composition (6 test pieces). On the same day of the preparation of adhesion test pieces, their shear bonding strength was measured on an Instron universal testing machine (Instron Model 5550) under the condition of the crosshead speed of 10 mm/min. The results are shown in Tables 1 to 3.

### Test Example 2

Some of the artificial nail compositions obtained, in Examples and Comparative examples were applied on user's own nails and their appearance was studied. The test, method was as follows.

### (1) Application

Practitioners authorized by Japan Nailist Association applied the artificial nail compositions to three subjects according to the following method, and adhesion durability and aesthetic appearance durability of the applied artificial nail compositions were evaluated.

Two practitioners carried out application to fingers of both hands (10 fingers) of five subjects. The application procedure was natural nail gel floater as the general application method, which followed the steps as follows:
(Step 1) Sand the surface of nails with a sponge nail file;
(Step 2) Clean off oil and dusts with a wiper soaked with a nail cleanser;
(Step 3) Apply the artificial nail composition of Example or Comparative example on the whole natural nail;
(Step 4) Carry out photo-polymerization for 20 seconds with a photo-polymerization apparatus (commercial product: Presto LED Lamp);
(Step 5) On the polymerized artificial nail composition, apply color using color gel (commercial product: Presto Color Gel);
(Step 6) Carry out photo-polymerization for 20 seconds with a photo-polymerization apparatus (commercial product: Presto LED Lamp);
(Step 7) Repeat the Step 5;
(Step 8) Repeat the Step 6;
(Step 9) Apply top gel (commercial product: Presto Top Gel) on the whole natural nail;
(Step 10) Carry out photo-polymerization for 20 seconds with a photo-polymerization apparatus (commercial product: Presto LED Lamp); and
(Step 11) Clean off uncured gel on the surface with a wiper soaked with a gel remover or cleanser.

### (2) Evaluation

In the context of the aesthetic appearance durability test, aesthetic appearance means the appearance which can be obtained by morphology formation (extension, shaping of nails) or drawing a design called nail art using the artificial nail composition of the present invention. In the aesthetic appearance test method, visual inspection of the artificial nails was carried out during everyday life after the application in order to evaluate the retention of the aesthetic appearance immediately after the application as well as detachment or loss of the artificial nail compositions from natural nails was determined.

The study was carried out for 14 days. Aesthetic appearance durability was defined, as a period from application to impairment of aesthetic appearance due to, for example, detachment or loss of the artificial nail compositions from natural nails. Longer aesthetic appearance durability is preferred. When superior aesthetic appearance was retained after the test period of 14 days, the aesthetic appearance durability was recorded as “14 days or more”. The results are shown in Tables 1 to 3. Meanings of the symbols A to C in the Tables are as follows.

A: No detachment or loss of the artificial nail composition was confirmed at 14 days after application for ail subjects.

B: Detachment or loss of the artificial nail composition was observed within 7 days after application for all subjects.

C: Detachment or loss of the artificial nail composition was observed within 3 days after application for ail subjects.

TABLE 1

| | Ex./Comp. Ex. | | | | | |
|---|---|---|---|---|---|---|
| | | | | | Comp. | Comp. |
| Component | Ex. 1 | Ex. 2 | Ex. 3 | Ex. 4 | Ex. 1 | Ex. 2 |

| | | | | | | | |
|---|---|---|---|---|---|---|---|
| Component (c) | PUDA | 65.0 | 65.0 | 65.0 | 65.0 | 65.0 | 65.0 |
| | IBMA | 8.0 | 13.0 | 18.0 | 23.0 | 28.0 | 17.0 |
| | TEGDMA | | | | | | |
| Component (a) | 2HEMA | 20.0 | 15.0 | 10.0 | 5.0 | | 15.0 |
| | 2HPMA | | | | | | |
| (a′) GM | | | | | | | |
| Component (b) | MA | 4.0 | 4.0 | 4.0 | 4.0 | 4.0 | |
| | 4META | | | | | | |
| | A-SA | | | | | | |
| | A-PCLA | | | | | | |
| Component (d) | MAPO | 2.0 | 2.0 | 2.0 | 2.0 | 2.0 | 2.0 |
| | BP | 0.5 | 0.5 | 0.5 | 0.5 | 0.5 | 0.5 |
| | 2HMPP | 0.5 | 0.5 | 0.5 | 0.5 | 0.5 | 0.5 |
| Total | | 100.0 | 100.0 | 100.0 | 100.0 | 100.0 | 100.0 |
| Adhesion | Average | 25.2 | 23.5 | 24.2 | 19.2 | 12.3 | 9.1 |
| strength | Standard | 1.3 | 4.0 | 1.2 | 4.0 | 4.3 | 3.1 |
| [MPa] | Deviation | | | | | | |

| | | | | |
|---|---|---|---|---|
| Aesthetic appearance | A: 14 | A: 14 | C: 3 | C: 3 days |
| durability | days | days or | days | |
| | or | more | | |
| | more | | | |

[Added amount: parts by weight]

Examples 1 to 4 shown in Table 1 are the artificial nail compositions consisting of the components (a), (b), (c) and (d). Comparative example 1 is the artificial nail composition consisting of the components (b), (c) and (d) without the component (a) and comparative example 2 is the artificial nail composition consisting of the components (a), (c) and (d) without the component (b).

As it is apparent from the adhesion strength shown in Table 1, Examples 1 to 4 have superior adhesion strength from 19.2 to 25.2 MPa, while comparative examples 1 and 2 showed low adhesion strength of 12.3 MPa and 9.1 MPa, respectively.

The results of aesthetic appearance durability obtained for Examples 1 and 4 were as sufficiently long as 14 days or more, as shown in Table 1, while the artificial nail compositions of Comparative examples 1 and 2 consisting of the components (b), (c) and (d) without the component (a) and of the components (a), (a) and (d) without the component, (b) respectively, have insufficient aesthetic appearance durability.

TABLE 2

| | | Ex./Comp. | | | | | | |
|---|---|---|---|---|---|---|---|---|
| | | | | | Comp. | Comp. | Comp. | Comp. |
| Component | | Ex. 5 | Ex. 6 | Ex. 7 | Ex. 3 | Ex. 4 | Ex. 5 | Ex. 6 |
| Component | PUDA | 65.0 | 65.0 | 65.0 | 65.0 | 65.0 | 65.0 | 65.0 |
| (c) | IBMA | 8.0 | 8.0 | 8.0 | 8.0 | 13.0 | 18.0 | 23.0 |
| | TEGDMA | 7.0 | 3.0 | 1.0 | | | | |
| Component | 2HEMA | 7.5 | 7.5 | 7.5 | | | | |
| (a) | 2HPMA | 7.5 | 7.5 | 7.5 | | | | |
| (a′) GM | | | | | 20.0 | 15.0 | 10.0 | 5.0 |
| Component | MA | 2.0 | 6.0 | 8.0 | 4.0 | 4.0 | 4.0 | 4.0 |
| (b) | 4META | | | | | | | |
| | A-SA | | | | | | | |
| | A-PCLA | | | | | | | |
| Component | MAPO | 2.0 | 2.0 | 2.0 | 2.0 | 2.0 | 2.0 | 2.0 |
| (d) | BP | 0.5 | 0.5 | 0.5 | 0.5 | 0.5 | 0.5 | 0.5 |
| | 2HMPP | 0.5 | 0.5 | 0.5 | 0.5 | 0.5 | 0.5 | 0.5 |
| Total | | 100.0 | 100.0 | 100.0 | 100.0 | 100.0 | 100.0 | 100.0 |
| Adhesion | Average | 20.0 | 21.3 | 23.7 | 2.9 | 2.8 | 2.9 | 2.9 |
| strength | Standard | 1.3 | 4.4 | 3.4 | 1.2 | 1.1 | 1.2 | 0.6 |
| [MPa] | Deviation | | | | | | | |
| Aesthetic | | | | A: 14 | C: 3 | | | C: 3 |
| appearance | | | | days or | days | | | days |
| durability | | | | more | | | | |

[Added amount: parts by weight]

Examples 5 to 7 shown in Table 2 are the artificial nail compositions consisting of the components (a), (b), (c) and (d). Comparative examples 3 to 6 are the artificial nail compositions consisting of the components (b), (c) and (d) without the component (a). The component (a′) used in Comparative examples 3 to 6 is a photo-polymerizabie monomer having one secondary alcoholic hydroxyl group in the molecule.

As it is apparent from the adhesion strength shown in Table 2, Examples 5 to 7 have superior adhesion strength from 20.0 to 23.7 MPa, while Comparative examples 3 to 6 show low adhesion strength from 2.3 to 2.9 MPa.

The result of aesthetic appearance durability obtained for Example 7 is as sufficiently long as 14 days or more, as shown in Table 2, while the artificial nail compositions of Comparative examples 3 and 6 comprising the components (b), (c) and (d) without the component (a) have insufficient aesthetic appearance durability.

TABLE 3

| | | Ex./Comp. | | | | | | | |
|---|---|---|---|---|---|---|---|---|---|
| | | | | Ex. | Ex. | Ex. | Ex. | Ex. | Ex. |
| Component | | Ex. 8 | Ex. 9 | 10 | 11 | 12 | 13 | 14 | 15 |
| Component | PUDA | 51.0 | 49.0 | 57.0 | 53.0 | 51.0 | 45.0 | 65.0 | 45.0 |
| (c) | IBMA | 10.0 | 10.0 | | | | 22.0 | 2.0 | 12.0 |
| | TEGDMA | | | | | | 10.0 | | 10.0 |
| Component | 2HEMA | 15.0 | 15.0 | 15.0 | 15.0 | 15.0 | 10.0 | 10.0 | 10.0 |
| (a) | 2HPMA | 15.0 | 15.0 | 15.0 | 15.0 | 15.0 | | | |
| (a′) GM | | | | | | | | | |
| Component | MA | | | 2.0 | 6.0 | 8.0 | | 10.0 | |
| (b) | 4META | 6.0 | 8.0 | 8.0 | 8.0 | 8.0 | | | |
| | A-SA | | | | | | 10.0 | 10.0 | |
| | A-PCLA | | | | | | | | 20.0 |
| Component | MAPO | 2.0 | 2.0 | 2.0 | 2.0 | 2.0 | 2.0 | 2.0 | 2.0 |
| (d) | BP | 0.5 | 0.5 | 0.5 | 0.5 | 0.5 | 0.5 | 0.5 | 0.5 |
| | 2HMPP | 0.5 | 0.5 | 0.5 | 0.5 | 0.5 | 0.5 | 0.5 | 0.5 |
| Total | | 100.0 | 100.0 | 100.0 | 100.0 | 100.0 | 100.0 | 100.0 | 100.0 |
| Adhesion | Average | 21.4 | 17.5 | 15.5 | 18.4 | 19.8 | 25.2 | 20.4 | 17.0 |
| strength | Standard | 1.3 | 2.7 | 3.2 | 2.9 | 4.4 | 2.9 | 5.3 | 3.3 |
| [MPa] | Deviation | | | | | | | | |

| | | | |
|---|---|---|---|
| Aesthetic appearance | A: 14 | A: | A: 14 |
| durability | days | 14 | days |
| | or | days | or |
| | more | or | more |
| | | more | |

[Added amount: parts by weight]

Examples 8 to 15 shown in Table 3 are the artificial nail compositions consisting of the components (a), (b), (c) and (d). As it is apparent from the adhesion strength shown in Table 3, Examples 8 to 15 have superior adhesion strength from 15.5 to 25.2 MPa.

The results of aesthetic appearance durability obtained for Examples 9, 11 and 14 are all as sufficiently long as 14 days or more, as shown in Table 3.

Collectively from the results shown in Tables 1 to 3, it is apparent that the present compositions have superior adhesion and thus superior aesthetic appearance durability compared to Comparative examples.

What is claimed is;

1. An artificial nail composition comprising:
a) a photo-polymerizable monomer having at least one primary alcoholic hydroxyl group in the molecule, and/or a photo-polymerizable monomer having at least one carboxyl group as well as at least one primary alcoholic hydroxyl group in the molecule;
(b) at photo-polymerizable monomer having at least one carboxyl group in the molecule except for the photo-polymerizable monomer (a);
(c) a photo-polymerizable monomer except for the photo-polymerizable monomers (a) and (b); and
(d) a photo-polymerization initiator.

2. The artificial nail composition according to claim 1, comprising 5 to 50 parts by weight of the photo-polymerizable monomer (a), 1 to 25 parts by weight of the photo-polymerizable monomer (b), 20 to 93.5 parts by weight of the photo-polymerizable monomer (c) and 0.5 to 5 parts by weight of the photo-polymerization initiator (d), the total of the components (a), (b), (c) and (d) being 100 parts by weight.

3. The artificial nail composition according to claim 1, which is liquid.

4. The artificial nail composition according to claim 1, which is free from an organic solvent,

5. The artificial nail composition according to claim 1, which is used for formation of an artificial nail on a surface of a natural nail.

6. The artificial nail composition according to claim 5, wherein a method for the formation comprises the steps of (1) applying the liquid artificial nail composition on the surface of the natural nail to form a coating film and (2) irradiating light to the coating film to polymerize the photo-polymerizable monomers.
